(19) United States
(12) Patent Application Publication
(10) Pub. No.: US 2021/0283145 A1
(43) Pub. Date: Sep. 16, 2021

(54) METHOD FOR THE TREATMENT OF VIRAL INFECTIONS

(71) Applicant: Softhale NV, Diepenbeek (BE)

(72) Inventors: Jürgen Rawert, Koln (DE); Jan-Torsten Tews, Munich (DE)

(21) Appl. No.: 16/907,336

(22) Filed: Jun. 22, 2020

Related U.S. Application Data
(60) Provisional application No. 62/989,414, filed on Mar. 13, 2020.

(51) Int. Cl. A61K 31/58; A61K 9/00; A61M 11/00

(52) U.S. Cl. CPC A61K 31/58; A61M 2250/00; A61M 11/007; A61K 9/0078

(57) ABSTRACT

The present invention provides for a method for the treatment of a respiratory disease, disorder, or condition resulting from a viral infection in a subject, such as a pulmonary viral infection, the method comprising the step of administering to said subject a medically active liquid in nebulized form by inhalation, wherein the medically active liquid comprises an inhalable corticosteroid (ICS) and wherein the medically active liquid is administered in nebulized form using an inhalation device.

### BACKGROUND OF THE INVENTION

The present invention relates to the field of methods for the treatment of viral infections, more specifically pulmonary viral infections such as infections by coronavirus. Furthermore, the present invention relates to the field of inhalation devices or the administration of medically active liquids for inhalation therapy. More specifically, the present invention relates to the administration of a medically active liquid comprising an inhalable corticosteroid (ICS) by inhalation.

Nebulizers or other aerosol generators for liquids are known from the art since a long time ago. Amongst others, such devices are used in medical science and therapy. There, they serve as inhalation devices for the application of active ingredients in the form of aerosols, i.e. small liquid droplets embedded in a gas. Such an inhalation device is known e.g. from document EP 0 627 230 B1. Essential components of this inhalation device are a reservoir in which the liquid that is to be aerosolized is contained; a pumping device for generation of a pressure being sufficiently high for nebulizing; as well as an atomizing device in the form of a nozzle. By means of the pumping device, the liquid is drawn in a discrete amount, i.e. not continuously, from the reservoir, and fed to the nozzle. The pumping device works without propellant and generates pressure mechanically.

WO 2018/19770 A1 discloses a soft mist inhalation devise (SMI) having an impingement type nozzle which has proven to be useful for the effective administration of pharmaceutically active liquids especially in cases in which the medically active liquid or, more specifically, the pharmaceutically active compound or ingredient contained therein has to be administered to the lungs of the patient or other subject in need thereof.

S. Matsuyama et al. have recently reported that the inhaled corticosteroid ciclesonide blocks coronavirus RNA replication by targeting the viral protein NSP15 (Non-Structural Protein 15) (doi: https://doi.org/10.1101/2020.0311.987016). The authors report that steroid compounds, which are expected to have dual functions in blocking host inflammation and coronavirus (e.g., MERS-CoV) replication, were screened from a chemical library. Within this library, ciclesonide, an inhaled corticosteroid, suppressed human coronavirus replication in cultured cells, but did not suppress replication of respiratory syncytial virus or influenza virus. The effective concentration of ciclesonide to block SARS-CoV-2 (the cause of COVID-19) replication (EC90) was 6.3 μM. After the eleventh consecutive coronavirus passage in the presence of ciclesonide, a resistant mutation was generated, which resulted in an amino acid substitution (A25V) in NSP15, as identified using reverse genetics. A recombinant virus with the mutation was also resistant to ciclesonide suppression of viral replication. These observations suggest that the effect of ciclesonide was specific to coronavirus, suggesting this is a candidate drug for treatment of patients suffering from human respiratory coronaviruses, including the SARS (severe acute respiratory syndrome) viruses, such as MERS and COVID-19.

It is thus an object of the present invention to provide a method for the effective administration of inhaled corticosteroids (ICS), especially ciclesonide in a convenient, effective and patient friendly manner that allows for the treatment of patients suffering from viral pulmonary infections such as COVID-19, especially in cases in which a large number of subjects has to be treated effectively. Further objects of the invention will be clear on the basis of the following description of the invention, examples and claims.

### SUMMARY OF THE INVENTION

In a first aspect, the invention relates to a method for the treatment of a respiratory disease, disorder, or condition resulting from a viral infection in a subject, the method comprising the step of administering to said subject a medically active liquid in nebulized form by inhalation, wherein the medically active liquid comprises an inhalable corticosteroid (ICS) and wherein the medically active liquid is administered in nebulized form using an inhalation device.

In a further aspect, the invention relates to a method for the treatment of a pulmonary viral infection in a subject, the method comprising the step of administering to said subject a medically active liquid in nebulized form by inhalation, wherein the medically active liquid comprises an inhalable corticosteroid (ICS) and wherein the medically active liquid is administered in nebulized form using an inhalation device.

In a further aspect, the present invention provides for the medically active liquid comprising an inhalable corticosteroid (ICS) for use in the treatment of a pulmonary viral infection in a subject, wherein the medically active liquid is administered in nebulized form using an inhalation device.

### BRIEF DESCRIPTION OF THE DRAWINGS

FIG. 1. Solubilities of selected inhalable corticosteroids in selected solvent systems.

FIG. 2. Average particle (drop) size distribution, entire spray duration, for ciclesonide in 70:30 ethanol-water via soft-mist inhaler nebulization, measured by laser diffraction (with 95% confidence interval based on T distribution).

FIG. 3. An embodiment of an inhalation device that may be used in the method of the present invention prior to its first use.

FIG. 4. An inhalation device similar to the one of FIG. 3, but without an outlet valve.

FIG. 5. The embodiment of FIG. 3, with a filled pumping chamber.

FIG. 6. The situation during the first actuation of the inhalation device of FIG. 3.

FIG. 7. The situation at the end of the first actuation.

FIG. 8. The situation after re-filling the pumping chamber.

### DETAILED DESCRIPTION OF THE INVENTION

The present invention provides for a method for the treatment of a respiratory disease, disorder, or condition resulting from a viral infection in a subject, the method comprising the step of administering to said subject a medically active liquid in nebulized form by inhalation, wherein the medically active liquid comprises an inhalable corticosteroid (ICS) and wherein the medically active liquid is administered in nebulized form using an inhalation device.

The present invention further provides for a method for the treatment of a pulmonary viral infection in a subject, the method comprising the step of administering to said subject a medically active liquid in nebulized form by inhalation, wherein the medically active liquid comprises an inhalable corticosteroid (ICS) and wherein the medically active liquid is administered in nebulized form using an inhalation device.

The medically active liquid to be administered by the method according to the present invention comprises an inhalable corticosteroid (ICS) which may be selected from a broad variety of corticosteroids that are suitable for inhalation or innovative administration to a subject, more specifically to a warm-blooded animal or human, especially to human in need thereof.

In specific embodiments, the inhalable corticosteroid may be selected from the group of inhalable corticosteroids consisting of prednisolone, prednisone, butixocort (e.g., butixocort propionate), flunisolide, beclomethasone, triamcinolone, budesonide, fluticasone, mometasone, ciclesonide, rofleponide, dexamethasone, etiprednol (e.g., etiprednol dichloroacetate), deflazacort, loteprednol, RPR-106541, NS-126 and ST-26.

More specifically, the inhalable corticosteroid to be administered by the method of the present invention may be selected from the group of corticosteroids consisting of beclomethasone, budesonide, fluticasone, mometasone and ciclesonide.

In especially preferred embodiments, the medically active liquid comprises ciclesonide as the inhalable corticosteroid. It should be noted however, that the inhalable corticosteroids as described above may be administered as the sole inhalable corticosteroid comprised by the medically active liquid or the form of a mixture of two or more of the inhalable corticosteroids as described above which may be comprised by the medically active liquid to be administered according to the present invention.

In further specific embodiments the chosen inhalable corticosteroid or mixture of two or more inhalable corticosteroids is administered to the lungs of the subject.

The method according to the present invention allows for a method for the treatment of a respiratory disease, disorder, or condition resulting from a viral infection in a subject. Additionally, the method allows for the treatment of the pulmonary viral infection in a patient or subject. Viral infections such as pulmonary viral infections that may be treated by the method according to the present invention may be selected from a broad variety of viral infections including coronavirus, influenza virus, rhinovirus, and adenovirus, such as SARS viruses, MERS viruses, H1N1 influenza, and Avian Flu H5N1. In specific embodiments, however, the pulmonary viral infection to be treated by the method of the present invention is an infection by a coronavirus. In some embodiments, the pulmonary viral infection is a lower respiratory tract infection (e.g., a pneumonia).

In further specific embodiments, the viral infection, e.g., pulmonary viral infection, to be treated by the method according to the present invention is a SARS-CoV-2 virus infection. Such SARS-CoV-2 virus infection it is believed to be the cause of the pandemic disease COVID-19. Accordingly, in specific embodiments, the method according to the present invention allows for the treatment of pulmonary viral infections in a subject or patient diagnosed with COVID-19.

The inhalable corticosteroid (ICS) to be administered by the method of the present invention may be administered to the subject in need thereof or patient in an amount of about 10 μg (mcg) to about 3000 μg (mcg) per day or from about 100 μg (mcg) to about 2000 μg (mcg) per day or from about 200 μg (mcg) to about 1000 μg (mcg) per day.

The medically active liquid or pharmaceutical composition to be administered by the method according to the invention is preferably formulated as a composition that is suitable, and adapted for inhalative use, in other words a composition that may be nebulized or atomized for inhalation and that is physiologically acceptable for inhalation by a subject.

The medically active liquid or pharmaceutical composition to be administered by inhalation according to the invention may be in the form of a dispersion, for example a suspension with a liquid continuous phase, and a solid dispersed phase or in the form of a solution.

In further embodiments, the medically active liquid or pharmaceutical composition may comprise, optionally, one or more physiologically acceptable excipients, which are suitable for inhalative use. Excipients which may be featured in the composition include, but are not limited to, one or more buffering agents to regulate or control pH of the solution, salts, taste-masking agents, surfactants, lipids, antioxidants, and co-solvents, which may be used to enhance or improve solubility; for example alcohols, specifically alcohols with 2 to 4, or preferably 2 or 3, carbon atoms, such as ethanol, propanol or iso-propanol or a glycol.

In specific embodiments, the medically active liquid to be nebulized and administered by the method according to the present invention comprises the inhalable corticosteroid and optionally the further pharmaceutically active ingredients or excipients dissolved in an alcoholic or aqueous liquid vehicle or solvent. In preferred embodiments, such liquid vehicle or solvent comprises water and/or ethanol, preferably ethanol. In further specific embodiments, such liquid vehicle or solvent comprises or preferably consists of ethanol or a mixture of ethanol and water, wherein ethanol may be comprised in an amount of at least about 50 wt.-%, or at least about 60 wt.-% or at least about 70 wt.-% or even more and ethanol in an amount of up to about 50 wt.-%, or up to about 40 wt.-% or up to about 30 wt.-% or less. In specific embodiments, the liquid vehicle or solvent comprises or consists of ethanol in an amount of about 60 to about 80 wt.-%, such as about 70 wt.-%, and water in an amount of about 40 to about 20 wt.-%, such as about 30 wt.-%.

In further specific embodiments, the medically active liquid to be administered by the method of the present invention may be essentially free of a propellant.

The inhalable corticosteroid or, more specifically, the medically active liquid to be administered according to the method of the present invention is usually administered in 1 to 4 doses per day, or 2 or 3 doses per day using an inhaler or inhalation device as described in further detail below.

In specific embodiments, the inhalable corticosteroid or, more specifically the medically active liquid comprising the inhalable corticosteroid and optionally the further pharmaceutically active components or excipients as described above may be administered for prolonged periods of time such as for seven weeks or even months, depending on severity and success of the treatment of the subject in need thereof. In further specific embodiments however the inhalable corticosteroid of the medically active liquid comprising such corticosteroid is preferably administered for a period of at least 5 days.

According to the present invention, the medically active liquid comprising the inhalable corticosteroid is administered to the subject in need of such administration by inhalation of the medically active liquid in nebulized form. Such nebulization and administration by inhalation can be performed using an inhalation device specifically handheld inhalation device. Suitable inhalation devices comprise soft-mist inhalers (SMIs). The specific embodiment of such a soft mist inhaler is described, e.g., in international patent application WO 2018/197730 A1, the contents of which are incorporated herein by reference in its entirety. It should be noted, however, that the inhaler device described therein is just one example of a suitable inhaler device to be used according to the present invention and, therefore should not be interpreted as limiting the scope of the invention in any respect.

Soft-mist inhalers as described above have been proven as a very effective means for providing medically active liquids or compositions or pharmaceutically active compounds contained therein into the lung of a patient or subject in need thereof. Such as soft mist inhaler comprises one or a plurality of impingement-type nozzles. Such an impingement-type nozzle is adapted to emit at least two jets of liquid which are directed such as to collide and break up into small aerosol droplets. The nozzle usually are firmly affixed to the user-facing side of the housing of the inhalation device in such a way that it is immobile, or non-moveable, relative to the housing or at least relative to the side or part of the housing which faces the user (e.g. patient) when the device is used.

In specific embodiments, the inhalation device that may be used to administer the medically active liquid comprising an inhalable corticosteroid is a hand-held inhalation device for delivering a nebulized medically active aerosol for inhalation therapy, comprising

(a) a housing having a user-facing side;

(b) an impingement-type nozzle for generating the nebulized aerosol by collision of at least two liquid jets, the nozzle being firmly affixed to the user-facing side of the housing such as to be immobile relative to the housing;

(c) a fluid reservoir arranged within the housing; and

(d) a pumping unit arranged within the housing, the pumping unit having
an upstream end that is fluidically connected to the fluid reservoir;
a downstream end that is fluidically connected to the nozzle;
   wherein the pumping unit is adapted for pumping fluid from the fluid reservoir to the nozzle;

wherein the pumping unit further comprises
(i) a riser pipe having an upstream end, wherein the riser pipe is
   adapted to function as a piston in the pumping unit, and
   firmly affixed to the user-facing side of the housing such as to be immobile relative to the housing; and
(ii) a hollow cylinder located upstream of the riser pipe, wherein the upstream end of the riser pipe is inserted in the cylinder such that the cylinder is longitudinally movable on the riser pipe;
(iii) a lockable means for storing potential energy when locked and for releasing the stored energy when unlocked, the means being arranged outside of, and mechanically coupled to, the cylinder such that unlocking the means results in a propulsive longitudinal movement of the cylinder towards the downstream end of the pumping unit.

Such a preferred inhalation device comprises a housing having a user-facing side, an impingement-type nozzle for generating the nebulized aerosol by collision of at least two liquid jets, a fluid reservoir arranged within the housing, and a pumping unit which is also arranged within the housing. The nozzle is firmly affixed to the user-facing side of the housing such as to be immobile relative to the housing. The pumping unit has an upstream end that is fluidically connected to the fluid reservoir and a downstream end that is fluidically connected to the nozzle. Furthermore, the pumping unit is adapted for pumping fluid from the fluid reservoir to the nozzle, and it comprises a riser pipe which is adapted to function as a piston in the pumping unit, a hollow cylinder and a lockable means for storing potential energy. The riser pipe is firmly affixed to the user-facing side of the housing such as to be immobile relative to the housing. The hollow cylinder is located upstream of the riser pipe, and the upstream end of the riser pipe is inserted in the cylinder such that the cylinder is longitudinally movable on the riser pipe. The lockable means is capable of storing potential energy when locked and adapted for releasing the stored energy when unlocked. The means is arranged outside of, and mechanically coupled to, the cylinder in such a way that unlocking the means results in a propulsive longitudinal movement of the cylinder towards the downstream end of the pumping unit.

As used herein, a hand-held inhalation device is a mobile device which can be conveniently held in one hand and which is suitable for delivering a nebulized medically active aerosol for inhalation therapy. In order to be suitable for inhalation therapy, the device must be able to emit a medically active aerosol whose particle size is respirable, i.e., small enough to be taken up by the lungs of a patient or user. Typically, respirable particles have a mass median aerodynamic diameter of not more than about 10 μm, in particular not more than about 7 μm, or not more than about 5 μm, respectively. In this respect, inhalation devices are substantially different from devices that emits spray for oral or nasal administration, such as disclosed in US 2004/0068222 A1.

The inhalation device that may be used in the method of the present invention is capable of delivering a nebulized aerosol. As used herein, an aerosol is a system having at least two phases: a continuous phase which is gaseous and which comprises a dispersed liquid phase in the form of small liquid droplets. Optionally, the liquid phase may itself represent a liquid solution, dispersion, suspension, or emulsion.

Important for the generation of a nebulized aerosol is a suitable nozzle. According to the invention, the nozzle is of the impingement type. This means that the nozzle is adapted to emit at least two jets of liquid which are directed such as to collide and break up into small aerosol droplets. The nozzle is firmly affixed to the user-facing side of the housing of the inhalation device in such a way that it is immobile, or non-moveable, relative to the housing or at least relative to the side or part of the housing which faces the user (e.g. patient) when the device is used.

The fluid reservoir which is arranged within the housing is adapted to hold or store the medically active liquid from which the nebulized aerosol is generated and delivered by the inhalation device.

The pumping unit which is also arranged within the housing is adapted to function as a piston pump, also referred to as plunger pump, wherein the riser pipe functions as the piston, or plunger, which is longitudinally moveable within the hollow cylinder. The inner segment of the hollow cylinder in which the upstream end of the riser pipe moves forms a pumping chamber which has a variable volume, depending on the position of the riser pipe relative to the cylinder.

The hollow cylinder which provides the pumping chamber is fluidically connected with the fluid reservoir, either directly or indirectly, such as by means of an optional reservoir pipe (or reservoir pipe section). Similarly, the riser pipe, whose reservoir-facing, interior (upstream) end which can be received in the hollow cylinder, is fluidically connected at its downstream or exterior end to the nozzle in a liquid-tight manner, either directly or indirectly.

In this context, the expression “hollow cylinder” refers to a part or member which is hollow in the sense that it comprises an internal void which has a cylindrical shape, or which has a segment having a cylindrical space. In other words, and as is applicable to other types of piston pumps, it is not required that the external shape of the respective part or member is cylindrical. Moreover, the expression “hollow cylinder” does not exclude an operational state of the respective part or member in which the “hollow” space may be filled with material, e.g., with a liquid to be nebulized.

As used herein, a longitudinal movement is a movement along the main axis of the hollow cylinder, and a propulsive movement is a movement of a part in a downstream (or forward) direction.

Importantly, the riser pipe of the pumping unit of the inhalation device of the invention is arranged downstream of the cylinder, and it is firmly affixed to the user-facing side of the housing such as to be immobile relative to the housing or at least to the part of the housing which comprises the user-facing side of the housing. For the avoidance of doubt, firmly fixed means either directly or indirectly (i.e. via one or more connecting parts) fixed such as to prevent relative movement between the respective parts. As the nozzle is also immobile relative to the housing or the respective part of the housing, the riser pipe is also immobile relative to the nozzle, and the pumping action is affected by the longitudinal movement of the hollow cylinder. A propulsive movement of the cylinder, which is arranged in an upstream position relative to the riser pipe, results in a decrease of the volume of the pumping chamber, and a repulsive movement of the cylinder results in an increase of the volume. In other words, the riser pipe maintains its position relative to the housing, and the hollow cylinder can alter its position relative to the housing, and in particular, along a longitudinal axis of the same, such as to perform a piston-in-cylinder-type movement of the immobile riser pipe in the moveable cylindrical member.

This arrangement differs from other impingement-type inhalation devices which rely on a pumping unit whose riser pipe is in an upstream position and a cylindrical member in a downstream position wherein the riser pipe is moveable and the cylindrical member is fixed to the housing, as disclosed in US 2012/0090603 A1. A key advantage of the described preferred inhalation device is that the passage between pumping chamber and fluid reservoir can be designed with less restrictions with respect to its dimensions. It is e.g. possible to accommodate a significantly larger inlet valve (also referred to as check valve), which is easier to manufacture since it does not have to be contained within a narrow riser pipe. Instead, the invention allows the use of a check valve whose size is only restricted by the interior size of the housing or the dimensions of the means for storing potential energy. In other words, the diameters of the valve, the riser pipe and—if used—the reservoir pipe do not need to match each other. Furthermore, since no movable piston needs to be connected to the fluid reservoir, the component which provides the fluid connection to the reservoir can be designed independently of the moveable component, i.e. the hollow cylinder, allowing the individual parts to be adapted to suit their respective individual functions. In this respect, the invention provides for higher design flexibility because the moveable hollow cylinder, due to its robust structure and dimensions, provides better opportunities for designing a mechanically stable connection with the reservoir than would a less robust moveable riser pipe. Also, the connection between the hollow cylinder and the fluid reservoir can be designed with a larger diameter, such that higher flow velocities and fluid viscosities become feasible. Further, a support for the reservoir can be integrated into any component that comprises the cylinder. Additionally, any vent for pressure equilibration of the reservoir can be moved away from the reservoir body itself to (e.g.) a connector which forms an interface between reservoir and hollow cylinder, thus facilitating construction and avoiding the necessity to provide an essentially “open” reservoir body.

As mentioned, the lockable means for storing potential energy is adapted to store energy in its locked state and to release the stored energy when unlocked. The means is mechanically coupled to the hollow cylinder in such a way such that unlocking the means results in a propulsive longitudinal movement of the cylinder towards the downstream end of the pumping unit. During this movement, the internal volume of the cylinder, i.e. the volume of the pumping chamber, decreases. Vice versa, when the means for storing potential energy is in the locked state, the hollow cylinder is in its most upstream position in which the volume of the pumping chamber is largest. The locked state could also be considered a primed state. When the state of the means for storing energy is altered from the unlocked to the locked state, which could be referred to as priming the device, the hollow cylinder performs a repulsive longitudinal movement, i.e., from its most downstream position towards its most upstream position. A pumping cycle consists of two subsequent and opposing movements of the cylinder starting from its most downstream position to its most upstream (or primed) position and—driven by the means for storing potential energy that now releases its energy—back to its most downstream position.

In one of the preferred embodiments of the described inhalation device, the pumping unit is a high-pressure pumping unit and adapted to operate, or to expel fluid, at a pressure of at least about 50 bar. In other preferred embodiments, the operating pressure of the pumping unit is at least about 10 bar, or at least about 100 bar, or from about 2 bar to about 1000 bar, or from about 50 bar to about 250 bar, respectively. As used herein, the operating pressure is the pressure at which the pumping unit expels fluid, in particular a medically active liquid, such as an inhalable aqueous liquid formulation of a pharmacologically active ingredient, from its pumping chamber in a downstream direction, i.e. towards the nozzle. In this context, the expression “adapted to operate” means that the components of the pumping unit are selected with respect to the materials, the dimensions, the quality of the surfaces and the finish are selected such as to enable operation at the specified pressure.

Moreover, such high-pressure pumping unit implies that the means for storing potential energy is capable of storing and releasing a sufficient amount of energy to drive the propulsive longitudinal movement of the cylinder with such a force that the respective pressure is obtained.

The means for the storage of potential energy may be designed as tension or pressure spring. Alternatively, besides a metallic or plastic body, also a gaseous medium, or magnetic force utilizing material can be used as means for energy storage. By compressing or tensioning, potential energy is fed to the means. One end of the means is supported at or in the housing at a suitable location; thus, this end is essentially immobile. With the other end, it is connected to the hollow cylinder which provides the pumping chamber; thus, this end is essentially moveable. The means can be locked after being loaded with a sufficient amount of energy, such that the energy can be stored until unlocking takes place. When unlocked, the means can release the potential energy (e.g. spring energy) to the cylinder with the pumping chamber, which is then driven such as to perform a (in this case, longitudinal) movement. Typically, the energy release takes place abruptly, so that a high pressure can build up inside the pumping chamber before a significant amount of liquid is emitted, which results in a pressure decrease. In fact, during a significant portion of the ejection phase, an equilibrium exists of pressure delivered by the means for the storage of potential energy, and the amount of already emitted liquid. Thus, the amount of liquid remains essentially constant during this phase, which is a significant advantage to devices which use manual force of the user for the emission, such as the devices disclosed in documents US 2005/0039738 A1, US 2009/0216183 A1, US 2004/0068222 A1, or US 2012/0298694 A1, since manual force depends on the individual user or patient and is very likely to vary largely during the ejection phase, resulting in inhomogeneous droplet formation, size, and amount. In contrast to the prior art, the means according to the invention ensures that the inhalation device delivers highly reproducible results.

The means for storing potential energy may also be provided in the form of a highly pressurized gas container. By suitable arrangement and repeatable intermittent activating (opening) of the same, part of the energy which is stored inside the gas container can be released to the cylinder. This process can be repeated until the remaining energy is insufficient for once again building up a desired pressure in the pumping chamber. After this, the gas container must be refilled or exchanged.

In one of the preferred embodiments, the means for storing potential energy is a spring having a load of at least 10 N in a deflected state. In a particularly preferred embodiment, the means for storing potential energy is a compression spring made of steel having a load from about 1 N to about 500 N in its deflected state. In other preferred embodiments, the compression spring from steel has a load from about 2 N to about 200 N, or from about 10 N to about 100 N, in its deflected state.

The inhalation device that may be used in the method of the present invention is preferably adapted to deliver the nebulized medically active aerosol in a discontinuous manner, i.e. in the form of discrete units, wherein one unit is delivered per pumping cycle. In this aspect, the device differs from commonly known nebulizers such as jet nebulizers, ultrasonic nebulizers, vibrating mesh nebulizers, or electrohydrodynamic nebulizers which typically generate and deliver a nebulized aerosol continuously over a period of several seconds up to several minutes, such that the aerosol requires a number of consecutive breathing maneuvers in order to be inhaled by the patient or user. Instead, the inhalation device of the invention is adapted to generate and emit discrete units of aerosol, wherein each of the units corresponds to the amount (i.e., volume) of fluid (i.e., medically active liquid) which is pumped by the pumping unit in one pumping cycle into the nozzle where it is immediately aerosolized and delivered to the user or patient. Vice versa, the amount of liquid pumped by the pumping unit in one pumping cycle determines the amount of the pharmacologically active agent which the patient receives per dosing. It is therefore highly important with respect to achieving the desired therapeutic effect that the pumping unit operates precisely, reliably and reproducibly. The inventors have found that the inhalation device incorporating the pumping unit as described herein is particularly advantageous in that it does exhibit high precision and reproducibility.

In one preferred embodiment, a single dose of the medication (i.e., of the nebulized aerosol of the medically active liquid) is contained in one unit, i.e., in the volume that is delivered from the pumping unit to the nozzle for aerosol generation in one single pumping cycle. In this case, the user or patient will prime and actuate the device only once, and inhale the released aerosol in one breathing maneuver, per dosing (i.e., per dosing event).

In another preferred embodiment, a single dose of the medication consists of two units of the aerosol, and thus requires two pumping cycles. Typically, the user or patient will prime the device, actuate it such as to release and inhale a unit of the aerosol, and then repeat the procedure. Alternatively, three or more aerosol units may constitute a single dosing.

The volume of fluid (e.g., of medically active liquid) that is pumped by the pumping unit in one pumping cycle is preferably in the range from about 2 to about 150 μl. In particular, the volume may range from about 0.1 to about 1000 μl, or from about 1 to about 250 μl, respectively. These volume ranges are nearly the same as the volume of liquid phase that is contained in one unit of aerosol generated by the inhalation device, perhaps with minor differences due to minute losses of liquid in the device.

In another preferred embodiment, the pumping unit comprises an inlet valve, also referred to as a check valve or inlet check valve, positioned in the hollow cylinder. According to this embodiment, the interior space of the hollow cylinder, i.e., the pumping chamber, is fluidically connected with the fluid reservoir via the inlet check valve. The inlet valve allows the inflow of liquid into the pumping chamber, but prevents the backflow of liquid towards, or into, the fluid reservoir. The position of the inlet valve may be at or near the upstream end of the cylinder such as to make nearly the entire internal volume of the hollow cylinder available for functioning as the pumping chamber. Alternatively, it may be more centrally located along the (longitudinal) main axis of the hollow cylinder such as to define an upstream segment and a downstream segment of the cylinder, the upstream segment being upstream of the inlet valve and the downstream segment being downstream of the valve. In this case the pumping chamber is located in the downstream segment.

As mentioned, one of the advantageous effects is that an inlet valve having relatively large dimensions may be accommodated in this position, i.e., at the upstream end of the pumping chamber. This is particularly beneficial as it allows for large dimensions of the fluid conduit(s) within the valve, thus enabling high fluid velocities which translate into a rapid filling of the pumping chamber during the priming of the inhalation device. Moreover, the use of liquids having a higher viscosity than ordinary liquid formulations for inhalation, such as highly concentrated solutions of soluble active ingredients, become feasible for inhalation therapy.

According to a further preferred embodiment, the inlet valve is adapted to open only when the pressure difference between the upstream and the downstream side of the valve, i.e. the fluid reservoir side and the pumping chamber side, is above a predefined threshold value, and remains closed as long as the pressure difference is below the threshold value. “Pressure difference” means that, irrespective of the absolute pressure values, only the relative pressure difference between the two sides is relevant for determining whether the valve blocks or opens. If, for example, the pressure on the upstream (reservoir) side is already positive (e.g., 1.01 bar due to thermal expansion), but the pressure on the downstream (pumping chamber) side is ambient pressure (1.0 bar, no activation of the device), the pressure difference (here: 0.01 bar) is below the threshold value (e.g., 20 mbar), which allows the valve to stay closed even when subject to a positive pressure in opening direction. This means that the check valve remains closed until the threshold pressure is met, thus keeping the passage between reservoir and pumping chamber safely shut e.g. when the inhalation device is not in use. Examples for threshold pressure differences are in the range of 1 to 1000 mbar, and more preferably between about 10 and about 500 mbar, or between about 1 and about 20 mbar.

When actuating the inhalation device, as the means for storing potential energy alters its state from a locked state to an unlocked state, energy is released which effects the cylinder to perform its propulsive longitudinal movement, significant pressure is built up in the pumping chamber. This generates a marked pressure difference (due to a high pressure in the pumping chamber and a substantially lower pressure in the fluid reservoir) which exceeds the threshold value of the pressure difference, so that the check valve opens and allows the pressure chamber to become filled with liquid from the reservoir.

A valve type that may be designed to operate with such a threshold pressure difference is a ball valve pre-loaded with a spring. The spring pushes the ball into its seat, and only if the pressure acting against the spring force exceeds the latter, the ball valve opens. Other valve types which—depending on their construction—may operate with such a threshold pressure difference are duckbill valves or flap valves.

The advantage of such a valve operating with a threshold pressure difference is that the reservoir can be kept closed until active use is being made of the inhalation device, thus reducing unwanted splashing of reservoir liquid during device transport, or evaporation during long-term storage of the device.

In a further preferred embodiment, the inhalation device that may be used in the method according to the invention further comprises an outlet valve inside the riser pipe, or at an end of the riser pipe, for avoiding a return flow of liquid or air from the riser pipe into the hollow cylinder. In many cases, the use of such outlet valve will prove to be advantageous. Typically, the downstream end of the riser pipe is located close to the nozzle. The nozzle is in fluidic communication with the outside air. After emitting, in aerosolized form, the amount of liquid which is delivered from the pumping unit through the nozzle, driven by the propulsive longitudinal movement of the cylinder, the pumping chamber must be refilled. For this purpose, it slides back on the riser pipe into its previous upstream position (i.e. performs a repulsive longitudinal movement), so that the interior volume of the pumping chamber increases. Along with this, a negative pressure (sometimes also referred to as “underpressure”) is generated inside the pumping chamber which causes liquid to be sucked into the pumping chamber from the fluid reservoir which is located upstream of the pumping chamber. However, such negative pressure may also propagate downstream through the riser pipe up to the outside of the nozzle and could lead to air being sucked into the device through the nozzle, or nozzle openings, respectively. This problem can be avoided by providing an outlet valve, also referred to as outlet check valve, which opens towards the nozzle openings and blocks in the opposite direction.

Optionally, the outlet valve is of a type that blocks below (and opens above) a threshold pressure difference as described in the context of the inlet valve above. If a ball valve with a spring is used, the spring force must be directed against the pumping chamber such that when the difference between the interior pressure of the pumping chamber and the ambient pressure exceeds the threshold pressure difference value, the outlet valve opens. The advantages of such a valve correspond to the respective aforementioned advantages.

As mentioned, the outlet valve may be positioned within the riser pipe. Alternatively, the inhalation device comprises an outlet valve which is not integrated within the riser pipe, but positioned at or near one of the ends of the riser pipe, in particular at or near its downstream end, e.g., in a separate connector between the riser pipe and the nozzle. This embodiment may be advantageous in certain cases, e.g., if there is a need for a riser pipe with a particularly small diameter which makes the integration of a valve difficult. By accommodating the outlet valve downstream of the riser pipe, a valve with a relatively large diameter may be used, thus simplifying the requirements for the valve design.

In a further alternative embodiment, the outlet valve is absent. This embodiment may be feasible as the fluid channels of an impingement-type nozzle may have relatively small cross sections, resulting in only minor or very slow back flow at the given pressure conditions during the priming of the device. If the amount of backflow is considered acceptable in view of a particular product application, the inhaler design may be simplified by avoiding the outlet valve.

In any case, whether the inhalation device is designed with or without an outlet valve, all other options and preferences described with respect to other device features are applicable to both of these alternative embodiments.

In a further preferred embodiment, the inhalation device according to the invention comprises a fluid reservoir which is firmly attached to the hollow cylinder such as to be moveable together with the hollow cylinder inside the housing. This means that in each ejection phase of the pumping cycle, the fluid reservoir moves together with the hollow cylinder from an initial (“upstream”) position, in which the pumping chamber has its maximum interior volume, towards an end (“downstream”) position, in which the volume of the pumping chamber is minimal; and during the subsequent “priming” step, the fluid reservoir returns together with the hollow cylinder to their initial (“upstream”) position.

As used herein, the expression “firmly attached” includes both permanent and non-permanent (i.e. releasable) forms of attachment. Moreover, it includes direct and indirect (i.e. via one or more connecting parts) types of attachment. At the same time, as mentioned above, “firmly attached” means that the respective parts are fixed to each other in such a way as to substantially prevent their movement relative to each other. In other words, two parts that are firmly attached to each other may only be movable together, and with respect to each other, they are non-movable or immobile.

One of the advantages of this embodiment wherein the fluid reservoir is firmly attached to the hollow cylinder is that it provides the smallest possible dead volume between the reservoir and the pumping chamber.

According to an alternative embodiment, the fluid reservoir is fluidically connected to the hollow cylinder by means of a flexible tubular element, and firmly attached to the housing. According to this embodiment, the reservoir is not firmly attached to the hollow cylinder and does not move along with it when the cylinder performs its longitudinal movements. Instead, it is firmly, but optionally detachably, directly or indirectly, attached to the housing or to a part of the housing. One advantage of this embodiment is that the energy which is abruptly released upon unlocking the means for storing potential energy solely acts on the hollow cylinder and not on the fluid reservoir. This may be particularly advantageous in cases in which the fluid reservoir in its initial (fully filled state) at the beginning of its usage has a relatively large mass which decreases overuse. A higher acceleration of the hollow cylinder would translate into a higher pressure in the pumping chamber.

For the avoidance of doubt, all other options and preferences described herein-above and below with respect to other device features are applicable to both of these alternatives, i.e., regardless of whether the fluid reservoir is firmly attached to the hollow cylinder or not.

In one embodiment, the fluid reservoir is designed to be collapsible, such as by means of a flexible or elastic wall. The effect of such design is that upon repeated use of the device which involves progressive emptying of the reservoir, the flexible or elastic wall buckles or folds such as to reduce the internal volume of the reservoir, so that the negative pressure which is necessary for extraction of a certain amount of liquid is not required to increase substantially over the period of use. In particular, the reservoir may be designed as a collapsible bag. The advantage of a collapsible bag is that the pressure inside the reservoir is almost independent of the filling level, and the influence of thermal expansion is almost negligible. Also, the construction of such a reservoir type is rather simple and already well established.

A similar effect can be achieved with a rigid container which has a moveable bottom (or wall) by means of which the interior volume of the reservoir can also be successively reduced.

As used herein, the term “medically active” refers to a compound which has pharmacologically activity which improves respiratory function and/or has an anti-viral effect (e.g., inhibiting the viral life cycle).

### DETAILED DESCRIPTION OF THE DRAWINGS

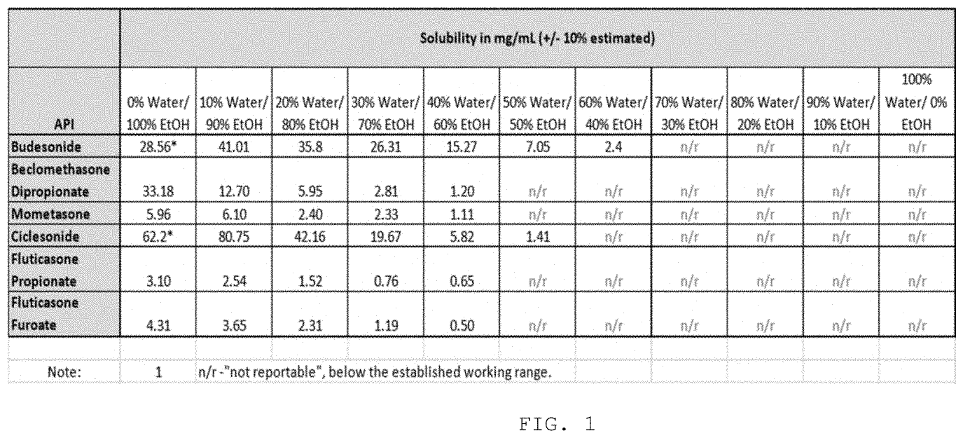

FIG. 1 shows the solubilities of various inhalable corticosteroids in various ethanolic solvent systems suitable for the inhalative administration by a soft-mist inhaler as described in Example 1.

FIG. 2. shows the average particle (drop) size distribution, entire spray duration, for ciclesonide in 70:30 ethanol-water via soft-mist inhaler nebulization, measured by laser diffraction (with 95% confidence interval based on T distribution) as described in Example 2.

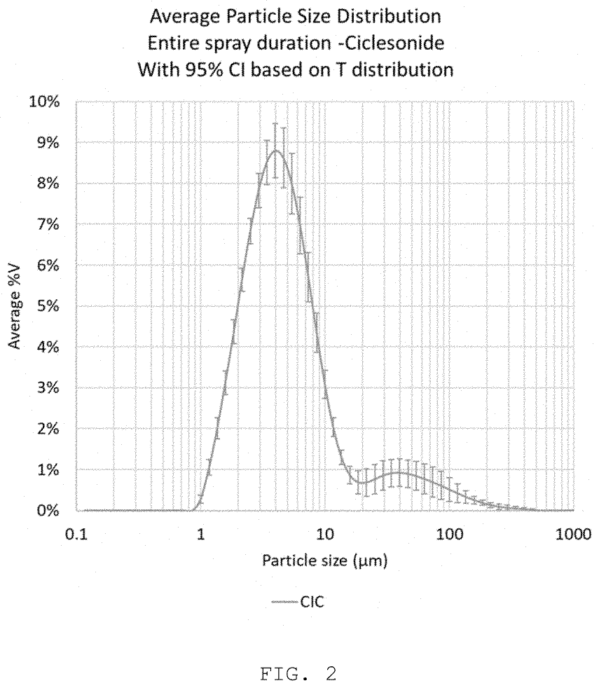

In FIG. 3, one of the preferred embodiments of an inhalation device useful for the method according to the present invention is depicted schematically and not-to-scale. FIG. 3 shows the situation prior to first use.

The inhalation device comprises a housing (1), which is preferably shaped and dimensioned such that it can be held with one hand and can be operated by one finger, e.g. a thumb or index finger (not shown). A fluid reservoir (2) for the storage of the medically active liquid (F) to be administered according to the present invention is located inside the housing (1). The depicted reservoir (2) is designed to be collapsible so that in the course of the emptying of the reservoir by the repeated use of the device, the soft or elastic walls deform such that the negative pressure required for withdrawing liquid from the reservoir remains substantially constant over time. A similar effect could be achieved with a rigid container that has a movable bottom by means of which the interior volume of the reservoir can also be successively be reduced (not shown).

Furthermore, the shown inhalation device comprises a pumping unit with a hollow cylinder (9) within the housing (1) which forms a pumping chamber (3) for the generation of the desired pressure which is necessary for emitting liquid (F) (i.e. the medically active liquid) and nebulising the same. The pumping unit may also comprise further components not depicted in the drawing, such as a push button, locking device, etc.

As a means for the storage of potential energy (7), a spring is provided which is coupled with one end (upwards directed, or downstream) to the cylinder (9) and which is supported at the housing (1) (lower part of the figure).

The shown inhalation device further comprises a riser pipe (5) with at least one reservoir-facing, or upstream, interior end (5A) which can be received in said cylinder (9). In other words, riser pipe (5) can be at least partially pushed into hollow cylinder (9), resulting in a decrease of the interior volume of pumping chamber (3). The term “interior volume” describes the volume of the space which extends from the reservoir-facing inlet of the cylinder (9) to the place where the interior end (5A) of the riser pipe (5) is located. In the depicted situation, riser pipe (5) is almost entirely contained in the cylinder (9). As a result, the interior volume of the pumping chamber (3), situated between inlet valve (4) and the interior end (5A) of riser pipe (5), is at a minimum.

Preferably, the section (or segment) of the hollow cylinder (9) which serves as, or accommodates, the pumping chamber (3) and which receives the riser pipe (5) exhibits a circular inner cross-section whose diameter relatively closely (e.g. except for a small gap) matches the diameter of the circular outer cross-section of the corresponding segment of the riser pipe (5). Of course, other (e.g. non-circular) cross section shapes are possible as well.

According to the depicted embodiment, inlet valve (4) is arranged between reservoir (2) and inlet of the pumping chamber (3) formed by the cylinder (9).

Furthermore, the inhalation device comprises a nozzle (6) which is connected liquid-tight to the exterior (or downstream) end (5B) of the riser pipe (5). Nozzle (6) is an impingement-type nozzle for generating the nebulised aerosol by collision of at least two liquid jets. Preferably, the cross sections of the liquid-containing channels are relatively small, typically in the region of microns.

Also depicted is an optional outlet valve (8) inside the riser pipe (5) for avoiding a backflow of liquid or air into the exterior end (5B) of the same from the outside. Outlet valve (8) is arranged in the interior end (5A) of riser pipe (5). Liquid (F) can pass outlet valve (8) in direction of nozzle (6), but outlet valve (8) blocks any undesired backflow in the opposite direction.

As can be seen in FIG. 3, riser pipe (5) is designed immobile with respect to the housing (1), and firmly attached to housing (1), indicated by the connection in the region of exterior end (5B) with housing (1). Riser pipe (5) is also firmly attached to nozzle (6), which, in turn, is attached to housing (1) as well. In contrast, the hollow cylinder (9) providing the pumping chamber (3) is designed to be moveable with respect to housing (1) and nozzle (6). The benefits of this design have been explained; reference is made to the respective sections of the description above.

Referring to FIG. 4, a device similar to the one of FIG. 3 is depicted. However, the embodiment shown in FIG. 4 lacks the (optional) outlet valve (8). All other components are present, and also the function is comparable. In this embodiment, pumping chamber (3) extends from downstream of the valve (4) up to nozzle (6), which is the location where the fluidic resistance increases significantly. In an alternative embodiment having a particularly small inner diameter of riser pipe (5), pumping chamber (3) extends only from downstream of the valve (4) up to upstream interior end (5A) of riser pipe (5).

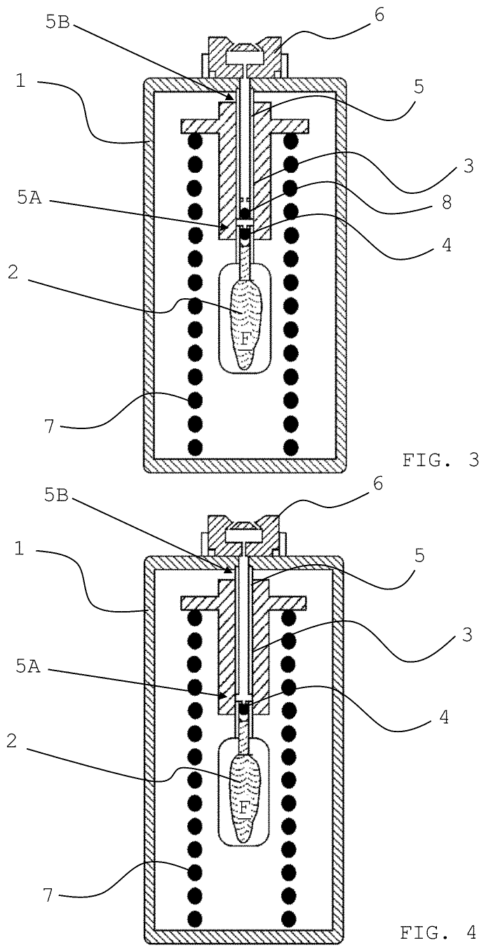

FIG. 5 shows the embodiment of FIG. 3 with a filled pumping chamber. The hollow cylinder (9) has been moved to its most upstream position, thereby loading the means for the storage of potential energy (7). Outlet valve (8) is closed due to negative pressure inside pumping chamber (3), and the inlet valve (4) is open towards the fluid reservoir (2). Increasingly collapsing walls of reservoir (2) allow the internal pressure in the reservoir (2) to remain nearly constant, while the pressure inside the pumping chamber (3) drops because of the propulsive longitudinal motion of the hollow cylinder (9), thus increasing the volume of pumping chamber (3). As a result, the pumping chamber (3) has been filled with the medically active liquid (F) from the reservoir (2).

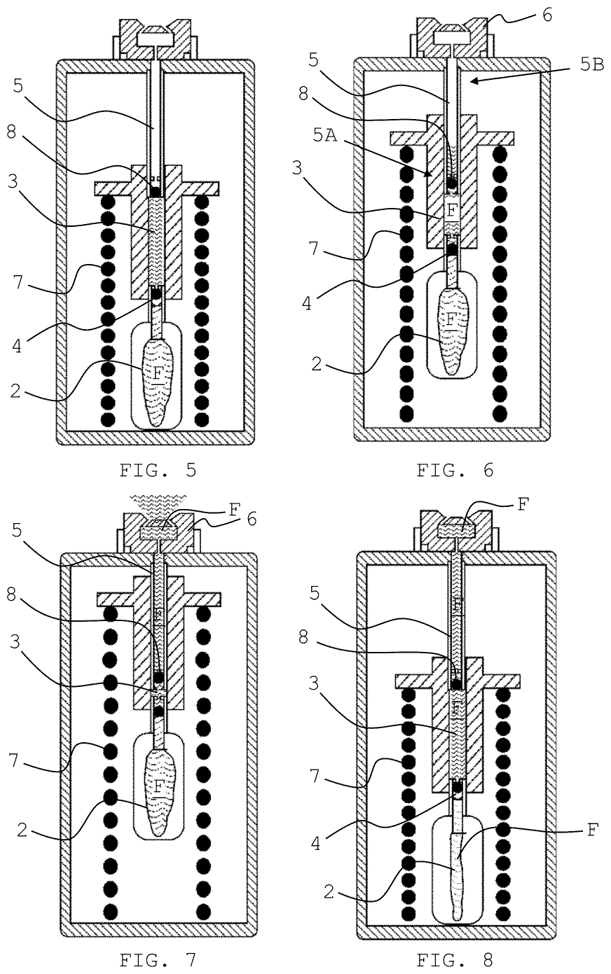

In FIG. 6, the situation after the first actuation of the inhalation device of FIG. 3 is shown. The means for the storage of potential energy (7) has been released from the loaded position as shown in FIG. 5. It pushes the cylinder (9) in a downstream direction such as to slide over the riser pipe (5). The interior end (5A) of the riser pipe (5) has come closer to the inlet check valve (4) which is now closed. As a result, the pressure inside the pumping chamber (3) rises and keeps the inlet valve (4) closed but opens outlet valve (8). Liquid (F) flows from the riser pipe (5) through its exterior end (5B) towards nozzle (6).

FIG. 7 shows the inhalation device of FIG. 3 in the situation at the end of the aerosol emission phase. The means for the storage of potential energy (7) is in its most relaxed end position (spring fully extended). Also, the hollow cylinder (9) has been pushed almost entirely onto riser pipe (5) such that the interior volume of pumping chamber (3) has reached its minimum. Most of the liquid (F) previously contained in the pumping chamber (3) has passed outlet valve (8) into the main segment of the riser pipe (5). Some liquid (F) has been pushed towards, and though, nozzle (6), where nebulisation takes place, such that a nebulised aerosol is emitted towards the user or patient.

In FIG. 8, the inhalation device of FIG. 3 in the situation after re-filling the pumping chamber is depicted. The hollow cylinder (9) has been moved (repulsively) in an upstream direction, thus increasing the volume of the pumping chamber (3) provided by the cylinder (9). The means for the storage of potential energy (7) has been loaded (spring compressed). During movement of cylinder (9) away from the nozzle (6), a negative pressure has been generated in the pumping chamber (3), closing outlet valve (8) and opening the inlet check valve 4. As a result, further liquid (F) is drawn from reservoir (2) into the pumping chamber (3). The inhalation device's pumping chamber (3) is filled again and ready for the next ejection of liquid (F) by releasing the spring.

### LIST OF REFERENCES

1 Housing
2 Fluid reservoir, reservoir
3 Pumping chamber
4 Inlet valve
5 Riser pipe
5A Interior end
5B Exterior end
6 Nozzle
7 Means for storing potential energy, means
8 Outlet valve
9 Hollow cylinder, cylinder
F Liquid, fluid, medically active liquid

The following examples serve to illustrate the invention, however should not to be understood as restricting the scope of the invention:

### EXAMPLES

### Example 1

FIG. 1 shows the solubilities of various inhalable corticosteroids in various ethanolic solvent systems suitable for the inhalative administration by a soft-mist inhaler.

The solutions prepared as above have been investigated for long-term stability and have proven to be stable over several months. Furthermore, the solutions prepared have been investigated for their suitability for administration using a soft mist inhaler. It was shown that the solutions were suitable for administration in nebulized form using a soft-mist inhaler.

### Example 2: Spray Tests Using a Solution of Ciclesonide (CIC) in Ethanol: Water (70:30) Via Nebulization by a Soft-Mist Inhaler

Table 1 shows the fractions of particle (droplet) sizes when the above described solution is nebulized with a soft-mist inhaler. ‘Event duration’ is the duration of the nebulization process in seconds (Stdev means standard deviation). ‘Dv10’ means the 10% of the overall number of particles has a mean diameter of the given value.

TABLE 1

CIC 70:30 EtOH:H₂O

| Formulation | Mean (n = 6) | Stdev |
|---|---|---|
| Event duration/s | 1.91 | 0.07 |
| Dv10/μm | 1.80 | 0.06 |
| Dv50/μm | 4.10 | 0.16 |
| Dv90/μm | 20.47 | 9.01 |

The resulting particle (droplet) size distribution as measured by laser diffraction is summarized in FIG. 2. The graph shows that the maximum of the particle (droplet) size distribution is below 5 μm which allows for good inhalability of the nebulized medically active liquid.

1. A method for the treatment of a respiratory disease, disorder, or condition resulting from a viral infection in a subject, wherein the viral infection is a severe acute respiratory syndrome (SARS), the method comprising the step of administering to said subject a medically active liquid comprising an aqueous solution essentially free of propellant in nebulized form by inhalation,
wherein the medically active liquid comprises ciclesonide, wherein the medically active liquid is administered in nebulized form using an inhalation device, wherein the inhalation device is a soft-mist-inhaler, wherein the ciclesonide is administered at a concentration of about 5 mg/mL to about 100 mg/mL, and wherein the subject is a human.

2. (canceled)

3. (canceled)

4. (canceled)

5. (canceled)

6. (canceled)

7. The method according to claim 1, wherein the ciclesonide is administered to the lungs of the subject.

8. (canceled)

9. (canceled)

10. The method according to claim 1, wherein the viral infection is a coronavirus infection.

11. The method according to claim 10, wherein the viral infection is a SARS-CoV-2 virus infection.

12. The method according to claim 11, wherein the subject is diagnosed with COVID-19.

13. The method according to claim 1, wherein the ciclesonide is administered in an amount of about 10 μg (mcg) to about 3000 μg (mcg) per day.

14. The method according to claim 1, wherein the ciclesonide is administered in 1 to 4 doses per day.

15. The method according to claim 1, wherein the ciclesonide is administered for a period of at least 5 days.

16. The method according to claim 1, wherein the soft-mist-inhaler used to administer the medically active liquid comprising ciclesonide is a hand-held device.

17. (canceled)

18. The method according to claim 1, wherein the soft-mist-inhaler has at least one impingement nozzle.

19. The method according to claim 1, wherein the soft-mist-inhaler used to administer the medically active liquid comprising ciclesonide is a hand-held inhalation device for delivering a nebulized medically active aerosol for inhalation therapy, comprising
(a) a housing having a user-facing side;
(b) an impingement nozzle for generating the nebulized aerosol by collision of at least two liquid jets, the nozzle being firmly affixed to the user-facing side of the housing to be immobile relative to the housing;
(c) a fluid reservoir arranged within the housing; and
(d) a pumping unit arranged within the housing, the pumping unit
having
an upstream end that is fluidically connected to the fluid reservoir;
a downstream end that is fluidically connected to the nozzle;
wherein the pumping unit is adapted for pumping fluid from the fluid reservoir to the nozzle;
wherein the pumping unit further comprises
(i) a riser pipe having an upstream end, wherein the riser pipe is
adapted to function as a piston in the pumping unit, and
firmly affixed to the user-facing side of the housing to be immobile relative to the housing; and
(ii) a hollow cylinder located upstream of the riser pipe, wherein the upstream end of the riser pipe is inserted in the cylinder such that the cylinder is longitudinally movable on the riser pipe;
(iii) a lockable means for storing potential energy when locked and for releasing the stored energy when unlocked, the means being arranged outside of, and mechanically coupled to, the cylinder such that unlocking the means results in a propulsive longitudinal movement of the cylinder towards the downstream end of the pumping unit.

20. The method according to claim 1, wherein the aqueous solution comprises a mixture of ethanol and water.

21. The method according to claim 20, wherein the mixture of ethanol and water is 70% wt. % ethanol and 30 wt. % water.
